(19) United States
(12) Patent Application Publication
(10) Pub. No.: US 2026/0276430 A1
(43) Pub. Date: Sep. 17, 2026

(54) REALTIME WATER SAMPLE AND SALINITY MEASUREMENT IN MULTIPHASE FLOW

(71) Applicant: SAUDI ARABIAN OIL COMPANY, Dhahran (SA)

(72) Inventors: Rodwan Zweit, Dhahran (SA); Mohammed Elmetwally Gomaa, Ras Tanura (SA); Abdulla Al Sarraf, Dhahran (SA)

(73) Assignee: SAUDI ARABIAN OIL COMPANY, Dhahran (SA)

(21) Appl. No.: 19/078,991

(22) Filed: Mar. 13, 2025

(51) Int. Cl. G01F 25/10; E21B 41/00; G01F 1/74; G01N 33/18; G01N 33/28

(52) U.S. Cl. CPC G01F 25/10; E21B 41/00; G01F 1/74; G01N 33/1886; G01N 33/2847

(57) ABSTRACT

A improved multiphase flow meter (MPFM) for performing real-time water sample and salinity measurement in multiphase flow of an oil and gas facility. The MPFM is connected to the multiphase flow in a flow line where a cavity is formed on a horizontal section of the flow line to capture a liquid sample under line pressure and temperature from the multiphase flow. Water salinity and conductivity of the captured liquid sample are measured in real-time to be used as a calibration reference for the MPFM to generate the water cut measurement. The salinity and conductivity measuring device and the MPFM collectively form a multiphase flow monitor. In particular, the multiphase flow monitor includes functionality to calibrate the water cut measurement in real-time.

### BACKGROUND

In oil and gas industry, water cut refers to the percentage of water in the produced fluids and is typically measured at the wellhead or within the production facility, specifically downstream of a separator. For example, after oil and water are separated in a production or test separator, a water cut meter can be installed on the oil outlet line to measure the water content in the oil stream. During well testing, a water cut meter can be used downstream of a two-phase test separator in the liquid leg to measure the water cut during the test. Water cut meters can be used to monitor individual wells and provide continuous real-time water cut data. Water cut meters can also be used to monitor group production lines as well as individual test lines at centralized production facilities. Water cut meters can also be installed on the discharge line from a tank to identify the rag layer (i.e., interface between oil and water) and monitor the dewatering process.

### SUMMARY

In general, in one aspect, the invention relates to a multiphase flow monitor that includes a multiphase flow meter installed on a flow line to generate a measurement of a multiphase flow in the flow line and a water sampling module protruding downward from a bottom side of the flow line in a horizontal section of the flow line. The water sampling module includes a bucket shaped cavity coupled to an interior volume of the flow line, the bucket-shaped cavity being configured to collect a water-only sample from the multiphase flow into the bucket shaped cavity, wherein non-water phase portion of the multiphase flow having lower density than water is repelled from the bucket shaped cavity due to a gravity effect, and a salinity probe coupled to the bucket shaped cavity, wherein the salinity probe generates a salinity measurement of the water sample to calibrate the multiphase flow meter to improve accuracy of the measurement of the multiphase flow.

In general, in one aspect, the invention relates to a system that includes an oil and gas facility comprising a pipeline network for transporting production fluids, a multiphase flow meter installed on a flow line in the pipeline network to generate a measurement of a multiphase flow of the production fluids in the flow line, and a water sampling module protruding downward from a bottom side of the flow line in a horizontal section of the flow line. The water sampling module includes a bucket shaped cavity coupled to an interior volume of the flow line where a water sample is collected from the multiphase flow into the bucket shaped cavity, wherein non-water phase portion of the multiphase flow having lower density than water is repelled from the bucket shaped cavity due to a gravity effect, and a salinity probe coupled to the bucket shaped cavity, wherein the salinity probe generates a salinity measurement of the water sample to calibrate the multiphase flow meter to improve accuracy of the measurement of the multiphase flow.

In general, in one aspect, the invention relates to a method to monitor a multiphase flow. The method includes installing a multiphase flow meter on a flow line to generate a measurement of a multiphase flow in the flow line, installing a water sampling module protruding downward from a bottom side of the flow line in a horizontal section of the flow line, the water sampling module including a bucket shaped cavity coupled to an interior volume of the flow line, and a salinity probe coupled to the bucket shaped cavity, collecting a water sample from the multiphase flow into the bucket shaped cavity, wherein non-water phase portion of the multiphase flow having lower density than water is repelled from the bucket shaped cavity due to a gravity effect, generating, using the salinity probe, a salinity measurement of the water sample, and calibrating the multiphase flow meter to generate a calibrated measurement of the multiphase flow.

Other aspects and advantages of the claimed subject matter will be apparent from the following description and the appended claims.

### BRIEF DESCRIPTION OF DRAWINGS

Specific embodiments of the disclosed technology will now be described in detail with reference to the accompanying figures. Like elements in the various figures are denoted by like reference numerals for consistency.

FIGS. 1A-1B show a system in accordance with one or more embodiments.

FIG. 2 shows a method flowchart in accordance with one or more embodiments.

FIGS. 3A-3B show implementation examples in accordance with one or more embodiments.

FIG. 4 shows a computing system in accordance with one or more embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (for example, first, second, third) may be used as an adjective for an element (that is, any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include a method and system for performing real-time water sample and salinity measurement in multiphase flow of an oil and gas facility. The method and system facilitate measuring water cut using a multiphase flow meter (MPFM) connected to the multiphase flow in a flow line. In one or more embodiments, a cavity is formed on a horizontal section of the flow line to capture a liquid sample under line pressure and temperature from the multiphase flow. Water salinity and conductivity of the captured liquid sample are measured in real-time to be used as a calibration reference for the MPFM to generate the water cut measurement. The salinity and conductivity measuring device and the MPFM collectively form a multiphase flow monitor. In one or more embodiments, the multiphase flow monitor includes functionality to calibrate the water cut measurement in real-time. In one or more embodiments, the salinity and conductivity measuring device, the MPFM, and the multiphase flow monitor are integrated into an automated system in an oil and gas facility to verify/calibrate multiphase flow metering performance in real time without human intervention.

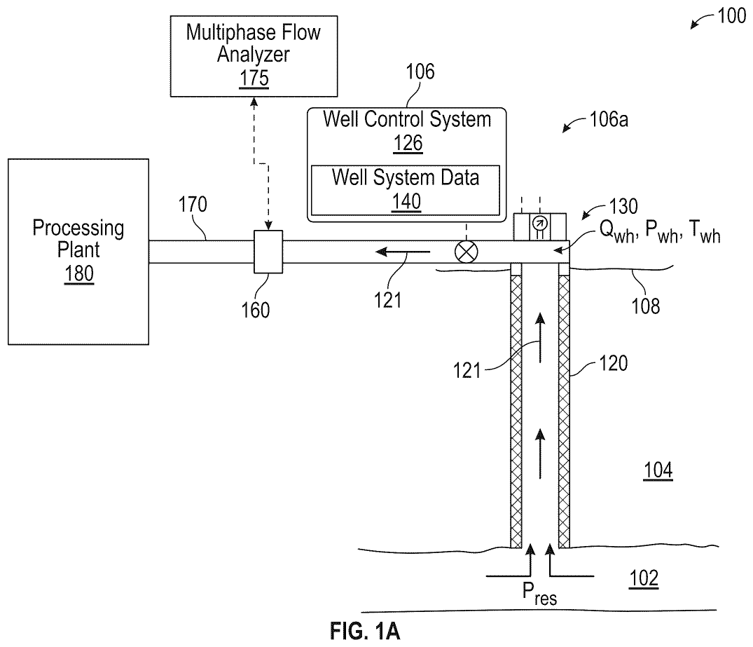

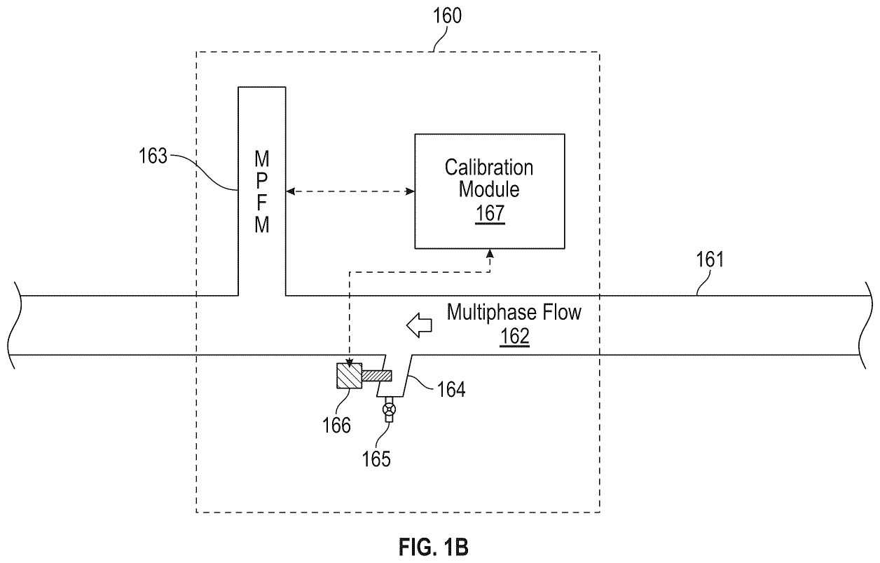

FIG. 1A shows a schematic diagram of an oil and gas facility in accordance with one or more embodiments. In one or more embodiments, one or more of the modules and/or elements shown in FIG. 1A may be omitted, repeated, combined and/or substituted. Accordingly, embodiments disclosed herein should not be considered limited to the specific arrangements of modules and/or elements shown in FIG. 1A.

As shown in FIG. 1A, FIG. 1A illustrates a well environment (100) that includes a hydrocarbon reservoir (“reservoir”) (102) located in a subsurface hydrocarbon-bearing formation (“formation”) (104) and a well system (106). The area where the well system (106) is located is referred to as a wellsite (106a). The hydrocarbon-bearing formation (104) may include a porous or fractured rock formation that resides underground, beneath the earth's surface (“surface”) (108). In the case of the well system (106) being a hydrocarbon well, the reservoir (102) may include a portion of the hydrocarbon-bearing formation (104). The hydrocarbon-bearing formation (104) and the reservoir (102) may include different layers of rock having varying characteristics, such as varying degrees of permeability, porosity, capillary pressure, and resistivity. In the case of the well system (106) being operated as a production well, the well system (106) may facilitate the extraction of hydrocarbons (or “production”) from the reservoir (102). The well system (106) may be part of a production system that further includes a pipeline network (170) and a processing plant (180) for transporting and processing the hydrocarbons, i.e., production from the reservoir (102).

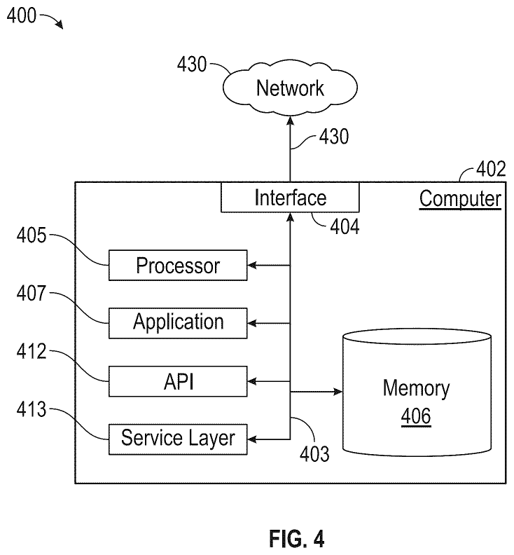

In some embodiments, the well system (106) includes a wellbore (120), a wellhead (130), and a well control system (“control system”) (126). The control system (126) may control various operations of the well system (106), such as well production operations, well completion operations, well maintenance operations, and reservoir monitoring, assessment and development operations. In some embodiments, the control system (126) includes a computer system that is similar to the computing system (400) described below with regard to FIG. 4 and the accompanying description.

The wellbore (120) may include a bored hole that extends from the surface (108) into a target zone of the hydrocarbon-bearing formation (104), such as the reservoir (102). The wellbore (120) may facilitate the circulation of drilling fluids during drilling operations, the flow of hydrocarbon production (“production”) (121) (e.g., oil and gas) from the reservoir (102) to the surface (108) during production operations, the injection of substances (e.g., water) into the hydrocarbon-bearing formation (104) or the reservoir (102) during injection operations, or the communication of monitoring devices (e.g., logging tools) into the hydrocarbon-bearing formation (104) or the reservoir (102) during monitoring operations (e.g., during in situ logging operations).

The wellhead (130) may include a rigid structure installed at the “up-hole” end of the wellbore (120), at or near where the wellbore (120) terminates at the Earth's surface (108). The wellhead (130) may include structures for supporting (or “hanging”) casing and production tubing extending into the wellbore (120). Production (121) may flow through the wellhead (130), after exiting the wellbore (120).

In some embodiments, during operation of the well system (106), the control system (126) collects and records well system data (140) for the well system (106). The well system data (140) may include, for example, a record of measurements of wellhead pressure (P_{wh}) (e.g., including flowing wellhead pressure), wellhead temperature (T_{wh}) (e.g., including flowing wellhead temperature), wellhead production rate (Q_{wh}) over some or all of the life of the well system (106), and water cut data. In some embodiments, the measurements and monitoring data are recorded in real-time, and are available for review, analysis, and/or use in real-time, i.e., within seconds, minutes or hours of the monitoring condition being sensed. In such an embodiment, the well system data (140) may be referred to as real-time well system data (140). Realtime well system data (140) may enable an operator of the well system (106) to assess a relatively current state of the well system (106), and make real-time decisions regarding development and maintenance of the well system (106) and the reservoir (102), such as on-demand adjustments in regulation of production flow from the well or preventive maintenance of equipment structures to prevent disruption to the production flow from the well.

In one or more embodiments, the processing plant (180) is an industrial process plant such as an oil/petroleum refinery where petroleum (crude oil) is transformed and refined, or other types of chemical processing plants. The processing plant (180) typically includes large, sprawling industrial complexes with extensive piping network running throughout, carrying streams or liquids between large chemical processing units, such as distillation columns. For example, the pipeline network (170) may extend throughout various portions of the processing plant (180). The processing plant (180) may include a control system similar to the well control system (126) to facilitate operations of the processing plant (180). For example, the control system of the processing plant (180) may collect and record water cut data throughout various portions of the processing plant (180) for facilitating the processing plant operations.

In some embodiments, one or more multiphase flow monitors (160) are installed throughout the well system (106) and/or the processing plant (180), e.g., at one or more locations of the pipeline network (170) to measure real-time water cut data as part of the well system data (140) and/or processing plant data. The multiphase flow monitor (160) is described in detail with reference to FIG. 1B below.

In some embodiments, the well system (106) and/or the processing plant (180) include a multiphase flow analyzer (175). For example, the multiphase flow analyzer (175) may include hardware and/or software with functionality to analyze the real-time water cut data for facilitating operations of the well system (106) and the processing plant (180). For example, the multiphase flow analyzer (175) may receive salinity/conductivity data and/or water cut data from multiple multiphase flow monitors (160) throughout the well system (106) and the processing plant (180) for analysis. The multiphase flow analyzer (175) may receive salinity/conductivity data and/or water cut data via wired and/or wireless data communications. For example, the multiphase flow analyzer (175) may communicate with one or more multiphase flow monitors (160) using the Modbus RTU protocol over an RS-485 interface or Ethernet connections for data transmission of flow rates, phase fractions, and other relevant parameters. Accordingly, the multiphase flow analyzer (175) may log and perform trend analysis of water cut data throughout the well system (106) and the processing plant (180) and generate a real-time alert in response to a pre-determined criterion. A real-time alert allows an operator to make real-time decisions regarding development and maintenance of the well system (106) and the processing plant (180).

While the multiphase flow analyzer (175) is shown at a well site, embodiments are contemplated where at least a portion of the multiphase flow analyzer (175) is located away from well sites. In some embodiments, at least a portion of the multiphase flow analyzer (175) is integrated with the well control system (126) and/or the control system of the processing plant (180). In some embodiments, the multiphase flow analyzer (175) includes a computer system that is similar to the computing system (400) described below with regard to FIG. 4 and the accompanying description.

FIG. 1B illustrates a multiphase flow monitor (160) depicted in FIG. 1A above. In one or more embodiments, one or more of the modules and/or elements shown in FIG. 1B may be omitted, repeated, combined and/or substituted. Accordingly, embodiments disclosed herein should not be considered limited to the specific arrangements of modules and/or elements shown in FIG. 1B.

As shown in FIG. 1B, the multiphase flow monitor (160) includes a multiphase flow meter (MPFM) (163) and a water sampling module (164) that are connected to a flow line (161). In one or more embodiments, the flow line (161) is a horizontal section of the pipeline network (170) depicted in FIG. 1A above. The water sampling module (164) has a hollow interior and includes a salinity/conductivity sensing probe (166) installed on side walls with a sealed bottom having a drain valve (165). The hollow interior is enclosed with the side walls and a bottom wall to form a cavity when the water sampling module (164) is attached to the bottom side of the flow line (161). The drain valve (165) is used to flush the cavity during cleaning or other maintenance procedures. The transversal cross-section of the cavity may have a polygonal, circular, elliptical, or other shape defined by the structure of the sidewalls. In this context, the cavity is referred to as a bucket shaped cavity. The bucket shaped cavity is coupled to an interior volume of the flow line (171) and captures a liquid sample under line pressure and temperature from the multiphase flow (162).

In one or more embodiments, the salinity/conductivity sensing probe (166) is installed on a permanent basis. To create the necessary cavity, one of two options may be employed. In one or more embodiments, the cavity can be created through hot-tapping, where a hole is drilled into the pipeline while it remains operational. Specialized equipment, e.g., tapping machines, may be used for this purpose, allowing for precise cuts to be made into the pipeline. In alternate embodiments, the cavity can be created by removing a section of the pipe (spool piece). Additionally, designing a sampling cavity on a horizontal line enables water accumulation and updating using gravity. This setup also incorporates salinity probe technology, specifically engineered to function effectively within liquids and environments containing minimal gas mixture.

In one or more embodiments, the cavity, or more specifically at least one sidewall, is slanted downward in the direction of the multiphase flow (162) to facilitate capturing the liquid sample under line pressure from the multiphase flow (162). For example, the slanted side wall and the flow line (161) may form an angle between 30 and 60 degrees. Non-water phase portion of the multiphase flow (162) having lower density than water is repelled from the cavity due to a gravity effect. The multiphase flow monitor (160) further includes a calibration module (167) that is coupled to the MPFM (163) and the salinity/conductivity sensing probe (166) via wired and/or wireless data connections, such as an RS-485 interface or Ethernet connections. The wired and/or wireless data connections are denoted as dashed line with double arrows in FIGS. 1A-1B. In one or more embodiments, the calibration module (167) receives salinity measurement data from the salinity/conductivity sensing probe (166) and sends it to the MPFM (163) as reference data for calibrating water cut measurements.

The MPFM (163) is a device used to measure the individual phase flow rates of constituent phases in the multiphase flow (162) where oil, water and gas mixtures are initially co-mingled together during the oil production processes. The MPFM (163) may be a commercially available MPFM that typically consists of a flow tube with integrated sensors such as electrical capacitance, gamma radiation, and differential pressure transducers, allowing it to measure the flow rate and composition of a mixed fluid stream (e.g., multiphase flow (162)) containing multiple phases (e.g., oil, water, and gas) simultaneously. The MPFM (163) measures the flow rate and composition of the multiphase flow (162) all within a single inline unit without the need for prior phase separation. In one or more embodiments, the MPFM (163) includes a sensing section with electrodes for electrical impedance measurements, a gamma ray source and detector for density determination, and a pressure differential measurement section to calculate flow velocity with the data from these sensors. In one or more embodiments, the MPFM (163) includes hardware and software with functionality to calculate the individual phase flow rates through pre-determined algorithms.

Commercially available MPFMs use reference water salinity to correct measured water cut reading. In an oil and gas facility, constant change of the produced water salinity necessitates MPFM calibrations with water salinity to ensure reliable water cut measurement. Commercially available salinity probes cannot accurately measure water salinity in three-phase fluids due to free gas influence on salinity measurement. In one or more embodiments, the bucket shaped water sampling module (164) installed on a horizontal line accumulates water via gravity from the multiphase flow (162) such that the salinity/conductivity sensing probe (166) operates in the liquid phase and low gas mix fluids. Commercially available salinity probes typically fail to provide accurate readings in three-phase environments, where the presence of free gas significantly impacts measurement accuracy. To address this limitation, the bucket shaped water sampling module (164) and the salinity/conductivity sensing probe (166) operate based on the principle of density separation where liquids settle at the bottom and gases rise to the top, thus ensuring that only water phases salinity is measured. In one or more embodiments, the principle of density separation is employed by utilizing a unique cavity design that allows water to accumulate and update, eliminating the interference caused by gas and providing reliable salinity readings. In this configuration, the water salinity is measured in one-phase (water) without being affected by free gas influence in three-phase fluids. That is, the water sampling module (164) accurately measures water salinity in multiphase (gas, oil & water) by accumulating only water in the cavity.

Accordingly, the salinity/conductivity sensing probe (166) precisely measures the total dissolved salt content of the water collected into the bucket shaped water sampling module (164). Measurement involves the transfer of an electrical current between two electrodes immersed in the collected water to measure electrical conductivity. In one or more embodiments, the calibration module (167) includes hardware and software with functionality to calibrate or otherwise adjust the measured water cut reading based on received salinity measurement data. In one or more embodiments, the salinity measurement data is received from the salinity/conductivity sensing probe (166) and inputted into the MPFM (163) by the calibration module (167) via an RS-485 interface or Ethernet connections.

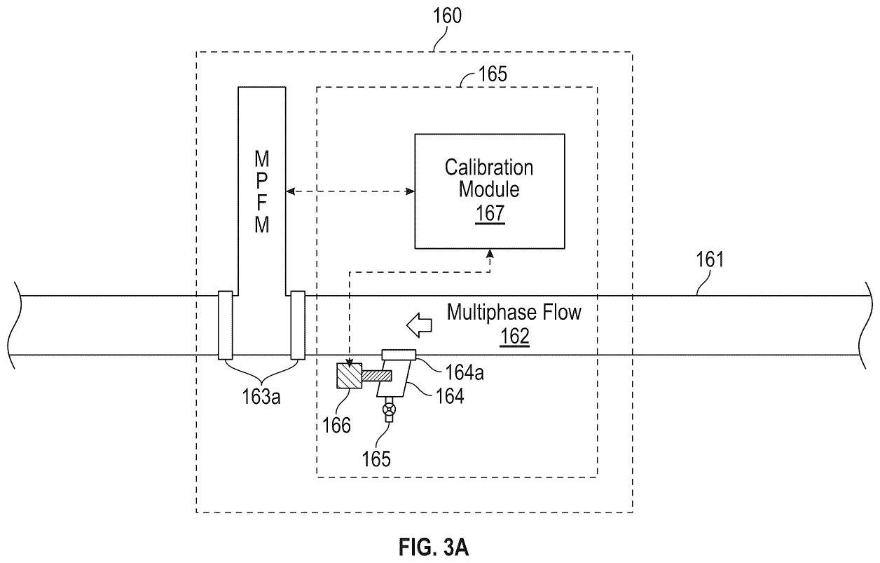

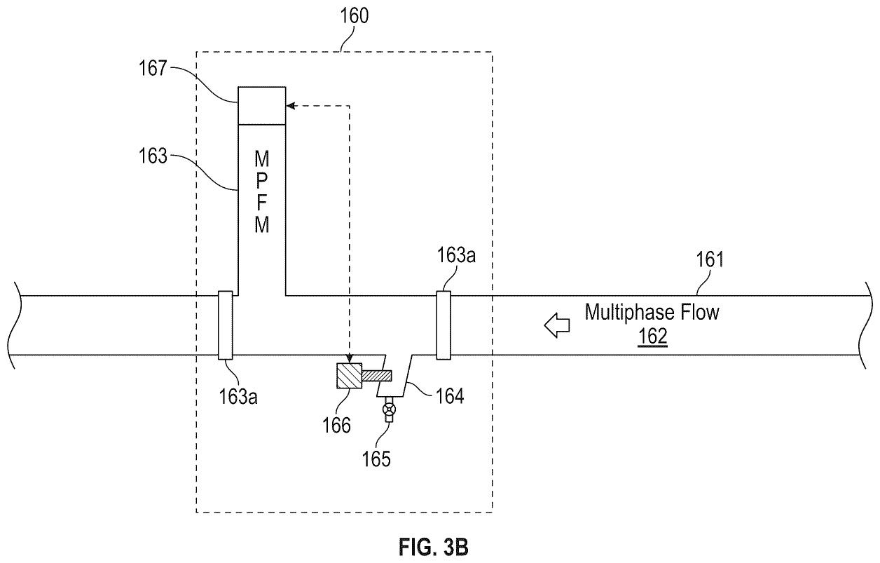

In one or more embodiments, the water sampling module (164) is installed onto the flow line (161) by a hot-tapping procedure or other suitable installation procedures. More details of installing the water sampling module (164) are described with reference to FIGS. 3A-3B below.

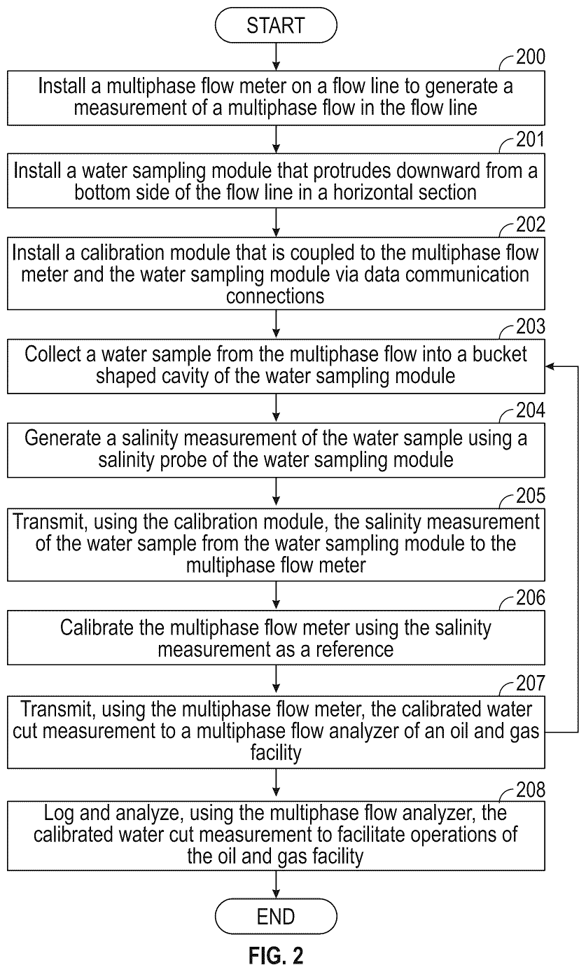

FIG. 2 shows a flowchart in accordance with one or more embodiments disclosed herein. One or more of the steps in FIG. 2 may be performed by the components of the well environment (100), in particular the multiphase flow monitor (160) and the multiphase flow analyzer (175), discussed above in reference to FIGS. 1A-1B. In one or more embodiments, one or more of the steps shown in FIG. 2 may be omitted, repeated, and/or performed in a different order than the order shown in FIG. 2. Accordingly, the scope of the disclosure should not be considered limited to the specific arrangement of steps shown in FIG. 2.

As noted above, multiphase flow meters use reference water salinity to correct measured water cut readings because salinity directly affects the electrical conductivity of the water, which is a key parameter in determining the water cut in a multiphase flow. In particular, multiphase flow meters often use dielectric or conductivity-based sensors to differentiate between oil, water, and gas phases. Accurate salinity information ensures the sensor correctly interprets the water phase. In many oil and gas operations, water salinity is not constant and can vary from one well to another or even within the same well over time due to changes in reservoir conditions, production methods, or external factors.

FIG. 2 shows the flowchart of a method to calibrate the multiphase flow meter in real-time to ensure accuracy, consistency, and reliability in the measurement of multiphase flows and facilitate effective operations of an oil and gas facility, such as a well system and/or processing facility.

Initially in Step 200, a multiphase flow meter is installed on a flow line to generate a measurement of a multiphase flow in the flow line. In one or more embodiments, the multiphase flow meter is installed onto the flow line using flanged connections. In one or more embodiments, the measurement of the multiphase flow includes a water cut measurement.

In Step 201, a water sampling module is installed that protrudes downward from a bottom side of the flow line in a horizontal section of the flow line. In one or more embodiments, the water sampling module includes a bucket shaped cavity coupled to an interior volume of the flow line and a salinity probe coupled to the bucket shaped cavity. In one or more embodiments, the water sampling module is installed onto the flow line via a hot tapping connection on the bottom side of the flow line.

In Step 202, a calibration module is installed that is coupled to the multiphase flow meter and the water sampling module via a data communication connection. In one or more embodiments, the water sampling module and the calibration module are integrated and installed onto the flow line as a single device. For example, the single device may be coupled to the flow line via a hot tapping connection on the bottom side of the flow line such that the bucket shaped cavity is coupled to the interior volume of the flow line through the hot tapping connection.

In one or more embodiments, the multiphase flow meter, the water sampling module, and the calibration module are integrated and installed onto the flow line as a single device. For example, the single device may be coupled to the flow line using flanged connections.

In Step 203, a water sample is collected from the multiphase flow into the bucket shaped cavity, where non-water phase portion of the multiphase flow having lower density than water is repelled from the bucket shaped cavity due to a gravity effect.

In Step 204, a salinity measurement of the water sample is taken using the salinity probe. That is, an accurate water salinity measurement is obtained in one-phase (water).

In Step 205, the salinity measurement of the water sample is transmitted, using the calibration module, from the water sampling module to the multiphase flow meter for calibrating the water cut measurement.

In Step 206, the multiphase flow meter is calibrated using the salinity measurement as a reference to improve accuracy of the measurement of the multiphase flow.

In Step 207, the calibrated water cut measurement is transmitted, using the multiphase flow meter, to a multiphase flow analyzer of an oil and gas facility where the multiphase flow monitor is installed. In one or more embodiments, Step 203 through Step 207 are iteratively performed, i.e., repeatedly performed to continuously obtain real-time calibrated water cut measurements.

In Step 208, the calibrated water cut measurement is logged and analyzed, using the multiphase flow analyzer, to facilitate operations of the oil and gas facility.

FIGS. 3A-3B show implementation examples of the multiphase flow monitor (160) depicted in FIG. 1B above. In one or more embodiments, one or more of the modules and/or elements shown in FIGS. 3A-3B may be omitted, repeated, combined and/or substituted. Accordingly, embodiments disclosed herein should not be considered limited to the specific arrangements of modules and/or elements shown in FIGS. 3A-3B.

The implementation examples described herein address the MPFM water cut measurement accuracy issue linked to constant change of produced water salinity, and time-consuming process to collect, analyze water sample and manually calibrate/update MPFM with reference water salinity. The implementation examples may be integrated into an automated system to verify multiphase flow monitoring performance in real-time with no human intervention.

As shown in FIG. 3A, the MPFM (163) of the multiphase flow monitor (160) includes flanges (163a) for connecting to the flow line (161). For example, the multiphase flow (162) is shut off while a section of the flow line (161) is removed to install the MPFM (163). The water sampling module (164) is connected to the flow line (161) via a hot tapping connection (164a) to form a cavity to capture real-time water sample for continuous water salinity measurement using the salinity/conductivity probe (166). Hot tapping is a procedure that allows a connection to be made to a pipeline or pressure vessel while still in use. The hot tapping connection (164a) is formed by (i) attaching a branch connection and valve to the outside of the flow line (161), (ii) cutting a hole in the flow line wall within the branch, (iii) removing the cut section of the flow line wall through the valve, and (iv) closing or opening the valve to control the hot tapping connection (164a).

Once connected to the flow line (161) via the hot tapping connection (164a), the water sampling module (164) measures accurate water salinity in multiphase (gas, oil & water) by accumulating only water in the cavity. Any water salinity changes, e.g., due to formation water connection with water injection, will be accounted for by the calibration module (167) calibrating the MPFM (163) using the accurate water salinity. As a result, time consuming manual collection of water sample for salinity analysis under hazard environment (e.g., due to H₂S) and manual inputting of reference salinity to MPFMs are eliminated. Operation safety is further improved by reducing operator exposure to radioactive sources equipped in some MPFMs.

In one or more embodiments, the calibration module (167), water sampling module (164), and the MPFM (163) are separate devices. In one or more embodiments, the MPFM (163) is a commercially available MPFM while the calibration module (167) and the water sampling module (164) are integrated in an enclosure as a single device (168). While the water sampling module (164) is shown as connected using the hot tapping connection (164a), it is contemplated that the single device (168) may connect to the flow line (161) using inline flanged connections in a similar manner as the MPFM (163).

FIG. 3B illustrates a further integrated example of the multiphase flow monitor (160). Similar to the example shown in FIG. 3A, the MPFM (163) includes flanges (163a) for connecting to the flow line (161). In contrast to the example shown in FIG. 3A, the water sampling module (164) and the calibration module (167) are integrated with the MPFM (163) as a single device that is connected to the flow line (161) via flanges (163a).

In summary, key features of the implementation examples shown in FIGS. 3A-3B include (i) accumulating water by gravity in a cavity, (ii) measuring accurate water salinity in one-phase (water) for live updating the MPFM, (iii) calibrating the MPFM using the accurate water salinity to measure accurate water cut, and (iv) eliminating manual water sampling and analysis, leading to an overall improvement in safety by reducing the exposure to Radioactive Sources equipped with some MPFMs.

Embodiments may be implemented on a computer system. FIG. 4 is a block diagram of a computer system (402) used to provide computational functionalities associated with described algorithms, methods, functions, processes, flows, and procedures as described in the instant disclosure, according to an implementation. The illustrated computer (402) is intended to encompass any computing device such as a high performance computing (HPC) device, a server, desktop computer, laptop/notebook computer, wireless data port, smart phone, personal data assistant (PDA), tablet computing device, one or more processors within these devices, or any other suitable processing device, including both physical or virtual instances (or both) of the computing device. Additionally, the computer (402) may include a computer that includes an input device, such as a keypad, keyboard, touch screen, or other device that can accept user information, and an output device that conveys information associated with the operation of the computer (402), including digital data, visual, or audio information (or a combination of information), or a GUI.

The computer (402) can serve in a role as a client, network component, a server, a database or other persistency, or any other component (or a combination of roles) of a computer system for performing the subject matter described in the instant disclosure. The illustrated computer (402) is communicably coupled with a network (430). In some implementations, one or more components of the computer (402) may be configured to operate within environments, including cloud-computing-based, local, global, or other environment (or a combination of environments).

At a high level, the computer (402) is an electronic computing device operable to receive, transmit, process, store, or manage data and information associated with the described subject matter. According to some implementations, the computer (402) may also include or be communicably coupled with an application server, e-mail server, web server, caching server, streaming data server, business intelligence (BI) server, or other server (or a combination of servers).

The computer (402) can receive requests over network (430) from a client application (for example, executing on another computer (402)) and responding to the received requests by processing the said requests in an appropriate software application. In addition, requests may also be sent to the computer (402) from internal users (for example, from a command console or by other appropriate access method), external or third-parties, other automated applications, as well as any other appropriate entities, individuals, systems, or computers.

Each of the components of the computer (402) can communicate using a system bus (403). In some implementations, any or all of the components of the computer (402), both hardware or software (or a combination of hardware and software), may interface with each other or the interface (404) (or a combination of both) over the system bus (403) using an application programming interface (API) (412) or a service layer (413) (or a combination of the API (412) and service layer (413). The API (412) may include specifications for routines, data structures, and object classes. The API (412) may be either computer-language independent or dependent and refer to a complete interface, a single function, or even a set of APIs. The service layer (413) provides software services to the computer (402) or other components (whether or not illustrated) that are communicably coupled to the computer (402). The functionality of the computer (402) may be accessible for all service consumers using this service layer. Software services, such as those provided by the service layer (413), provide reusable, defined business functionalities through a defined interface. For example, the interface may be software written in JAVA, C++, or other suitable language providing data in extensible markup language (XML) format or other suitable format. While illustrated as an integrated component of the computer (402), alternative implementations may illustrate the API (412) or the service layer (413) as stand-alone components in relation to other components of the computer (402) or other components (whether or not illustrated) that are communicably coupled to the computer (402). Moreover, any or all parts of the API (412) or the service layer (413) may be implemented as child or sub-modules of another software module, enterprise application, or hardware module without departing from the scope of this disclosure.

The computer (402) includes an interface (404). Although illustrated as a single interface (404) in FIG. 4, two or more interfaces (404) may be used according to particular needs, desires, or particular implementations of the computer (402). The interface (404) is used by the computer (402) for communicating with other systems in a distributed environment that are connected to the network (430). Generally, the interface (404) includes logic encoded in software or hardware (or a combination of software and hardware) and operable to communicate with the network (430). More specifically, the interface (404) may include software supporting one or more communication protocols associated with communications such that the network (430) or interface's hardware is operable to communicate physical signals within and outside of the illustrated computer (402).

The computer (402) includes at least one computer processor (405). Although illustrated as a single computer processor (405) in FIG. 4, two or more processors may be used according to particular needs, desires, or particular implementations of the computer (402). Generally, the computer processor (405) executes instructions and manipulates data to perform the operations of the computer (402) and any algorithms, methods, functions, processes, flows, and procedures as described in the instant disclosure.

The computer (402) also includes a memory (406) that holds data for the computer (402) or other components (or a combination of both) that can be connected to the network (430). For example, memory (406) can be a database storing data consistent with this disclosure. Although illustrated as a single memory (406) in FIG. 4, two or more memories may be used according to particular needs, desires, or particular implementations of the computer (402) and the described functionality. While memory (406) is illustrated as an integral component of the computer (402), in alternative implementations, memory (406) can be external to the computer (402).

The application (407) is an algorithmic software engine providing functionality according to particular needs, desires, or particular implementations of the computer (402), particularly with respect to functionality described in this disclosure. For example, application (407) can serve as one or more components, modules, applications, etc. Further, although illustrated as a single application (407), the application (407) may be implemented as multiple applications (407) on the computer (402). In addition, although illustrated as integral to the computer (402), in alternative implementations, the application (407) can be external to the computer (402).

There may be any number of computers (402) associated with, or external to, a computer system containing computer (402), each computer (402) communicating over network (430). Further, the term “client,” “user,” and other appropriate terminology may be used interchangeably as appropriate without departing from the scope of this disclosure. Moreover, this disclosure contemplates that many users may use one computer (402), or that one user may use multiple computers (402).

In some embodiments, the computer (402) is implemented as part of a cloud computing system. For example, a cloud computing system may include one or more remote servers along with various other cloud components, such as cloud storage units and edge servers. In particular, a cloud computing system may perform one or more computing operations without direct active management by a user device or local computer system. As such, a cloud computing system may have different functions distributed over multiple locations from a central server, which may be performed using one or more Internet connections. More specifically, cloud computing system may operate according to one or more service models, such as infrastructure as a service (IaaS), platform as a service (PaaS), software as a service (SaaS), mobile “backend” as a service (MBaaS), serverless computing, artificial intelligence (AI) as a service (AIaaS), and/or function as a service (FaaS).

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A multiphase flow monitor, comprising:
a multiphase flow meter installed on a flow line to generate a measurement of a multiphase flow in the flow line; and
a water sampling module protruding downward from a bottom side of the flow line in a horizontal section of the flow line, the water sampling module comprising:
a bucket shaped cavity coupled to an interior volume of the flow line, the bucket-shaped cavity being configured to collect a water-only sample from the multiphase flow into the bucket shaped cavity, wherein non-water phase portion of the multiphase flow having lower density than water is repelled from the bucket shaped cavity due to a gravity effect; and
a salinity probe coupled to the bucket shaped cavity, wherein the salinity probe generates a salinity measurement of the water sample to calibrate the multiphase flow meter to improve accuracy of the measurement of the multiphase flow.

2. The multiphase flow monitor of claim 1,
wherein the measurement of the multiphase flow comprises a water cut measurement.

3. The multiphase flow monitor of claim 2, further comprising:
a calibration module coupled to the multiphase flow meter and the water sampling module via a data communication connection, wherein the calibration module transmits the salinity measurement of the water sample from the water sampling module to the multiphase flow meter for calibrating the water cut measurement.

4. The multiphase flow monitor of claim 3,
wherein the multiphase flow monitor is installed in an oil and gas facility,
wherein the multiphase flow meter transmits the calibrated water cut measurement to a multiphase flow analyzer of the oil and gas facility, and
wherein the multiphase flow analyzer logs and analyzes the calibrated water cut measurement to facilitate operations of the oil and gas facility.

5. The multiphase flow monitor of claim 1,
wherein the water sampling module is coupled to the flow line via a hot tapping connection on the bottom side of the flow line, and
wherein the bucket shaped cavity is coupled to the interior volume of the flow line through the hot tapping connection.

6. The multiphase flow monitor of claim 5,
wherein the water sampling module and the calibration module are integrated into a single device.

7. The multiphase flow monitor of claim 1,
wherein the multiphase flow meter, the water sampling module, and the calibration module are integrated into a single device.

8. A system, comprising:
an oil and gas facility comprising a pipeline network for transporting production fluids;
a multiphase flow meter installed on a flow line in the pipeline network to generate a measurement of a multiphase flow of the production fluids in the flow line; and
a water sampling module protruding downward from a bottom side of the flow line in a horizontal section of the flow line, the water sampling module comprising:
a bucket shaped cavity coupled to an interior volume of the flow line where a water sample is collected from the multiphase flow into the bucket shaped cavity, wherein non-water phase portion of the multiphase flow having lower density than water is repelled from the bucket shaped cavity due to a gravity effect; and
a salinity probe coupled to the bucket shaped cavity, wherein the salinity probe generates a salinity measurement of the water sample to calibrate the multiphase flow meter to improve accuracy of the measurement of the multiphase flow.

9. The system of claim 8,
wherein the measurement of the multiphase flow comprises a water cut measurement.

10. The system of claim 9, further comprising:
a calibration module coupled to the multiphase flow meter and the water sampling module via a data communication connection, wherein the calibration module transmits the salinity measurement of the water sample from the water sampling module to the multiphase flow meter for calibrating the water cut measurement.

11. The system of claim 10, further comprising a multiphase flow analyzer that
receives the calibrated water cut measurement from the multiphase flow monitor, and
logs and analyzes the calibrated water cut measurement to facilitate operations of the oil and gas facility.

12. The system of claim 8,
wherein the water sampling module is coupled to the flow line via a hot tapping connection on the bottom side of the flow line, and
wherein the bucket shaped cavity is coupled to the interior volume of the flow line through the hot tapping connection.

13. The system of claim 12,
wherein the water sampling module and the calibration module are integrated into a single device.

14. The system of claim 8,
wherein the multiphase flow meter, the water sampling module, and the calibration module are integrated into a single device.

15. A method to monitor a multiphase flow, comprising:
installing a multiphase flow meter on a flow line to generate a measurement of a multiphase flow in the flow line;
installing a water sampling module protruding downward from a bottom side of the flow line in a horizontal section of the flow line, the water sampling module comprising:
a bucket shaped cavity coupled to an interior volume of the flow line; and
a salinity probe coupled to the bucket shaped cavity;
collecting a water sample from the multiphase flow into the bucket shaped cavity, wherein non-water phase portion of the multiphase flow having lower density than water is repelled from the bucket shaped cavity due to a gravity effect;
generating, using the salinity probe, a salinity measurement of the water sample; and
calibrating the multiphase flow meter to generate a calibrated measurement of the multiphase flow.

16. The method of claim 15,
wherein the measurement of the multiphase flow comprises a water cut measurement.

17. The method of claim 16, further comprising:
installing a calibration module coupled to the multiphase flow meter and the water sampling module via a data communication connection; and
transmitting, using the calibration module, the salinity measurement of the water sample from the water sampling module to the multiphase flow meter for calibrating the water cut measurement.

18. The method of claim 17, further comprising:
installing a multiphase flow analyzer in an oil and gas facility where the multiphase flow monitor is installed;
transmitting, using the multiphase flow meter, the calibrated water cut measurement to the multiphase flow analyzer; and
logging and analyzing, using the multiphase flow analyzer, the calibrated water cut measurement to facilitate operations of the oil and gas facility.

19. The method of claim 15,
wherein the water sampling module and the calibration module are integrated into a single device,
wherein the single device is coupled to the flow line via a hot tapping connection on the bottom side of the flow line, and
wherein the bucket shaped cavity is coupled to the interior volume of the flow line through the hot tapping connection.

20. The method of claim 15,
wherein the multiphase flow meter, the water sampling module, and the calibration module are integrated into a single device.
